(19) United States
(12) Patent Application Publication
(10) Pub. No.: US 2026/0345449 A1
(43) Pub. Date: Nov. 5, 2026

(54) COSMETIC

(71) Applicant: SHISEIDO COMPANY, LTD., Tokyo (JP)

(72) Inventors: Yudai MATSUZAKI, Tokyo (JP); Yusuke OKUYAMA, Tokyo (JP)

(73) Assignee: SHISEIDO COMPANY, LTD., Tokyo (JP)

(21) Appl. No.: 19/545,006

(22) Filed: Feb. 19, 2026

(30) Foreign Application Priority Data
Mar. 4, 2025 (JP) 2025-033437

(51) Int. Cl. A61K 8/34; A61Q 19/00; A61Q 19/02

(52) U.S. Cl. CPC A61K 8/347; A61K 8/345; A61Q 19/007; A61Q 19/02

(57) ABSTRACT

The present invention provides a cosmetic having a high moisturizing effect when applied to skin, and at the same time, having improved feel during use, such as a permeation feeling, a slimy feeling, or a squeaky feeling. The cosmetic according to the present invention comprises (A) a bakuchiol and (B) polyglycerin such as diglycerin.

### CROSS-REFERENCE TO RELATED APPLICATIONS

### Background of the Invention

### Field of the Invention

The present invention relates to a cosmetic that exhibits a high moisturizing effect when applied to skin and is also excellent in feel during use such as permeation feeling at the same time.

### Background Art

It is known that a polyhydric alcohol such as glycerin exerts a moisturizing effect by being applied to a cosmetic. Therefore, it is often blended into an external preparation for skin such as cosmetics or cleansers. However, in cosmetics or the like containing glycerin, insufficient properties may be exhibited in terms of feel during use at the time of use, for example, occurrence of sliminess, stickiness, or the like, or poor permeation feeling. When polyglycerin such as diglycerin or triglycerin is used instead of glycerin, the moisturizing effect is improved, but the deterioration of feel during use tends to increase. For this reason, there is a trade-off relationship between the moisturizing effect by polyglycerin and the feel during use, and it may be difficult to increase the blending amount of polyglycerin. Therefore, there has been a demand for development of a cosmetic excellent in feel during use while achieving a sufficient moisturizing effect.

On the other hand, various agents are blended in cosmetics or the like in order to improve the skin condition. As such agents, various agents have been studied, and for example, use of retinol as an agent having a whitening effect or a bakuchiol having an effect similar thereto has been studied.

### CITATION LIST

### Patent Literature

Patent Literature 1: JP 2022-079822 A

### SUMMARY OF THE INVENTION

According to the present invention, a cosmetic having a high moisturizing effect when applied to skin, and at the same time, having improved feel during use, such as a permeation feeling, a slimy feeling, or a squeaky feeling.

According to the present invention, the following invention is provided.

[1]

A cosmetic including:
(A) a bakuchiol; and
(B) polyglycerin.
   [2]

The cosmetic according to [1], further including: (C) an alkyl-modified carboxyvinyl polymer.

[3]

The cosmetic according to [1] or [2], further including: (D) a polysaccharide.

[4]

The cosmetic according to any one of [1] to [3], further including: (E) a carboxamide derivative.

[5]

The cosmetic according to any one of [1] to [4], in which component (B) comprises diglycerin.

[6]

The cosmetic according to any one of [2] to [5], in which component (C) comprises an (acrylate/alkyl acrylate (C10-30)) crosspolymer.

[7]

The cosmetic according to any one of [3] to [6], in which component (D) comprises xanthan gum.

[8]

The cosmetic according to any one of [4] to [7], in which the component (E) comprises allantoin.

[9]

The cosmetic according to any one of [1] to [8], in which a mass ratio B/A of a content amount of component (B) to a content amount of component (A) contained in the cosmetic is 0.01 to 100.

[10]

The cosmetic according to any one of [2] to [9], in which a ratio C/A of a content amount of component (C) to a content amount of component (A) contained in the cosmetic is 0.01 to 20. [11]

The cosmetic according to any one of [1] to [10], which is an oil-in-water cosmetic.

According to the present invention, a cosmetic excellent in moisturizing effect and feel during use is provided. The cosmetic according to the present invention can achieve both a high moisturizing effect mainly brought about by polyglycerin, and excellent feel during use in which deterioration of permeation feeling often impaired by polyglycerin and an increase in slimy feeling or squeaky feeling are suppressed.

### DETAILED DESCRIPTION OF THE INVENTION

A cosmetic according to the present invention (hereinafter, sometimes referred to simply as a “cosmetic”) comprises:
(A) a bakuchiol; and
(B) polyglycerin. Each of these components and components that can be additionally blended in will be described below.

### (A) Bakuchiol

The cosmetic according to the present invention contains a bakuchiol (hereinafter, sometimes referred to as component (A)). The bakuchiol is a terpenophenol compound represented by the following Formula (a):

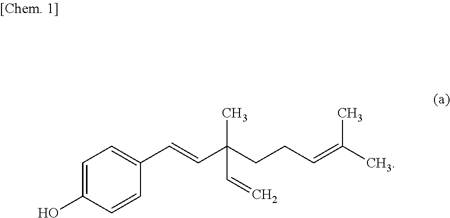 [SMILES: C=CC(C)(/C=C/c1ccc(O)cc1)CCC=C(C)C]

The bakuchiol can be obtained from a natural product or can be prepared by synthesis, and a production method is not particularly limited, and can be obtained, for example, by extraction from babchi seed. It is known that the bakuchiol has an antioxidant action and an anti-inflammatory action, and exhibits an effect similar to that of retinol conventionally used as a whitening agent. The bakuchiol that can be used in the present invention includes derivatives and salts thereof in addition to the bakuchiol itself.

In the present invention, component (A) can also obtain a whitening effect peculiar to bakuchiol, and also has an unexpected effect that the feel during use can be improved, when combined with polyglycerin described later. Although the mechanism by which these effects are obtained is not clear, component (A) exhibits an effect of significantly reducing the deterioration of the feel during use caused by polyglycerin.

In the present invention, a content of component (A) can be adjusted according to a content of polyglycerin described later, and is, for example, preferably 0.001 to 5 mass %, and more preferably 0.01 to 3 mass %, based on a total mass of the cosmetic.

### (B) Polyglycerin

The cosmetic according to the present invention comprises polyglycerin (hereinafter, referred to as component (B)). In the present invention, polyglycerin refers to a condensate of glycerin, and refers to a condensate of a dimer or more of glycerin. Polyglycerins are known to comprise a condensate having a degree of polymerization of 30 or more, and although there is no particular upper limit, polyglycerins having a degree of polymerization of 10 or less are preferred from the viewpoint of moisturizing effect and feel during use. Diglycerin, which is a dimer, and triglycerin, which is a trimer, are more preferred, and diglycerin is particularly preferred. In the present invention, component (B) can be used in combination of two or more kinds of polyglycerins, and in this case, component (B) preferably contains diglycerin.

Here, diglycerin is represented by Formula (b-1). Triglycerin is a trimer formed by dehydration condensation of three glycerin molecules, which are bonded in a linear, cyclic or branched chain, and can have the structures of Formulae (b-2-1) to (b-2-5).

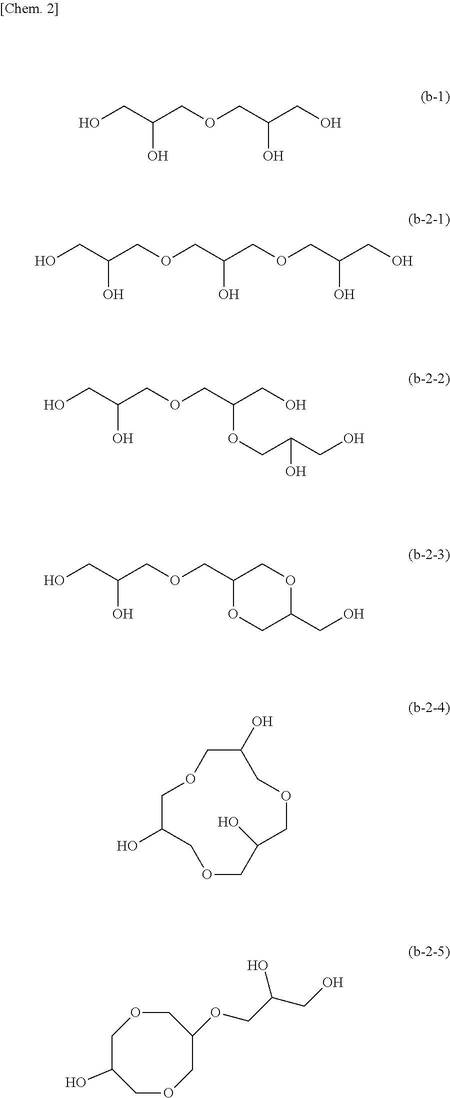 [SMILES: OCC(O)COCC(O)CO ; OCC(O)COCC(O)COCC(O)CO ; OCC(O)COCC(CO)OCC(O)CO ; OCC(O)COCC1COC(CO)CO1 ; OC1COCC(O)COCC(O)COC1 ; OCC(O)COC1COCC(O)COC1]

In the present invention, the content of component (B) is preferably not less than a certain content in order to obtain a sufficient moisturizing effect, and is preferably not more than a certain content in order to maintain a feel during use at a preferable content. Specifically, the content of component (B) is preferably 0.01 to 10 mass % and more preferably 0.1 to 7 mass % based on the total mass of the cosmetic.

In addition, in the present invention, it is preferable to consider the balance of the content amounts of components (A) and (B) in order to achieve both the moisturizing effect obtained when the cosmetic is used and the feel during use.

Specifically, a mass ratio B/A of the content amount of component (B) to the content amount of component (A) contained in the cosmetic is preferably 0.01 to 100, and more preferably 0.1 to 50.

### (C) Alkyl-Modified Carboxyvinyl Polymer

The cosmetic according to the present invention comprises the above-described components (A) and (B) as essential components, but may further contain other components as necessary. One of these other components is an alkyl-modified carboxyvinyl polymer (hereinafter, referred to as component (C)). Component (C) further improves the permeation feeling when the cosmetic according to the present invention is used, and exhibits an effect of further suppressing the squeaky feeling.

Component (C) is an alkyl acrylate methacrylate copolymer, and is a polymer in which at least a part of carboxyl groups of a polymer having acrylic acid or methacrylic acid as a main chain is esterified with an alkyl group. The alkyl group bonded by an ester bond may be linear or branched, and the number of carbon atoms is preferably 10 to 30. Component (C) is preferably a crosspolymer in which a polymer or copolymer containing alkyl acrylate or alkyl methacrylate as a polymerization unit is crosslinked with each other. Examples of component (C) include acrylate/alkyl acrylate (C10-30) crosspolymer. In the present invention, component (C) can be used by combining two or more kinds of alkyl-modified carboxyvinyl polymers, and in this case, component (C) preferably contains an acrylate/alkyl acrylate (C10-30) crosspolymer.

The content of component (C) is preferably 0.01 to 1 mass % and more preferably 0.05 to 0.5 mass % with respect to the total mass of the cosmetic.

In addition, a ratio C/A of the content amount of component (C) to the content amount of component (A) contained in the cosmetic is preferably 0.01 to 20, and more preferably 0.03 to 10, in order to realize excellent permeation feeling.

### (D) Polysaccharide

The cosmetic according to the present invention can comprise a polysaccharide (hereinafter, referred to as component (D)) as other components.

In the present invention, as component (D), any polysaccharide generally used in cosmetics and cleansers can be selected and used. Specifically, as component (D), xanthan gum, succinoglycan, gellan gum, pectin, alginate, propylene glycol alginate, hydroxyethylcellulose, hydroxypropylcellulose, hydroxypropyl methylcellulose, hyaluronic acid or a salt thereof, acetylated hyaluronic acid or a salt thereof, and the like can be used. Among them, water-soluble polysaccharides derived from microorganisms such as xanthan gum, succinoglycan, and gellan gum are preferable, and xanthan gum is particularly preferable. As component (D), two or more kinds of polysaccharides can be used in combination, and in this case, it is preferable to contain xanthan gum.

In general, component (D) is often used for adjusting a viscosity of a composition, and exhibits an effect of suppressing a slimy feeling when the cosmetic is used in the present invention. In order to sufficiently exhibit such an effect, the content of water-soluble polysaccharide of component (D) is preferably 0.005 to 2 mass % and more preferably 0.01 to 1.5 mass % with respect to the total mass of the cosmetic.

### (E) Carboxamide Derivative

The cosmetic according to the present invention can comprise a carboxamide derivative (hereinafter, referred to as component (E)) as other components.

The carboxamide derivative can be selected from those commonly used in cosmetics and cleansers depending on the purpose, and examples thereof include allantoin, 1-(2-hydroxyethyl)-2-imidazolidinone, 1-(2-hydroxyethyl)-2-pyrrolidone, urea, and ethyleneurea. Among them, allantoin is preferably used because the moisturizing effect is greatly improved, and at the same time, the effect of suppressing the slimy feeling at the time of use is great. Component (E) may be a combination of three or more carboxamide derivatives, and in this case, it is preferable to comprise allantoin.

The content of component (E) can be adjusted depending on the purpose, and is preferably 0.001 to 2 mass % and more preferably 0.005 to 1 mass % based on the total mass of the cosmetic.

The cosmetic according to the invention can further comprise water. As the water, water used for cosmetic products, quasi-drugs, and the like can be used. For example, purified water, ion-exchanged water, and tap water can be used.

The cosmetic according to the present invention can also contain an oil component. Examples of the oil component include hydrocarbon oil, higher fatty acid, higher alcohol, ester oil, and silicone oil.

Examples hydrocarbon oil include isododecane, isohexadecane, isoparaffin, mineral oil (liquid paraffin), paraffin, ceresin, and squalene.

Examples of the higher fatty acid include carboxylic acids having 10 or more carbon atoms.

Examples of the higher alcohol include a linear alcohol and a branched alcohol.

Examples of the ester oil include esters of higher fatty acids and higher alcohols.

Examples of the silicone oil include chain polysiloxanes such as dimethylpolysiloxane, methylphenylpolysiloxane, and diphenylpolysiloxane, and cyclic polysiloxanes such as octamethylcyclotetrasiloxane, decamethylcyclopentasiloxane, and dodecamethylcyclohexasiloxane. In addition, silicone oil having a vinyl group at the terminal or having a Si—H group can also be used.

The cosmetic according to the present invention can take any form such as an aqueous cosmetic, an oily cosmetic, an oil-in-water cosmetic, or a water-in-oil cosmetic by appropriately containing these water or oil components. Among them, the present invention is preferably an oil-in-water cosmetic.

In addition to the above components, the cosmetic according to the present invention may contain other components usually used for cosmetic products and pharmaceuticals. Examples of such components include the following components, and one kind or two or more kinds can be blended as long as the effects of the present invention are exhibited.

When the cosmetic according to the present invention is an emulsified cosmetic, a surfactant can be blended as a component for stabilizing a dispersion state thereof. The surfactant can be used by being arbitrarily selected from nonionic surfactants, anionic surfactants, cationic surfactants, and amphoteric surfactants, which are usually used for external preparations such as cosmetics and quasi-drugs.

The cosmetic according to the present invention contains polyglycerin exhibiting a moisturizing effect, and can contain polyhydric alcohols such as polyethylene glycol, propylene glycol, dipropylene glycol (DPG), and 1,3-butylene glycol (BG) as other moisturizing agents.

In addition, a lower alcohol, a thickener other than polysaccharides, a sequestering agent, a neutralizing agent, a pH adjusting agent, various agents, an ultraviolet absorber, a powder component, a fragrance, and the like can also be appropriately blended as components.

Examples of the cosmetic according to the present invention include skin care cosmetics (for example, lotion, emulsion, cream, serum, pack, mask, and the like), makeup cosmetics (for example, foundation, makeup base, and the like), skin cleansers (for example, facial cleanser, makeup remover, and the like), sunscreen cosmetics, and ointments. These forms are merely examples, and the cosmetic according to the present invention is not limited to these forms.

### EXAMPLES

The present invention will be specifically described based on the following examples, but the present invention is not limited to these examples. The content is represented in mass % with respect to the total amount, unless otherwise specified.

### Examples 1 to 11 and Comparative Example 1

Cosmetics of examples and comparative example were prepared with the formulation shown in Table 1. Furthermore, regarding the performance of these cosmetics, evaluation was performed as follows.

### [Evaluation of Usability: Permeation Feeling]

The cosmetic prepared above was applied to the skin by ten expert panelists and permeation feeling after the application was evaluated. The evaluation criteria are as follows.
A+: Ten panelists evaluated as having permeation feeling.
A: Eight to nine panelists evaluated as having permeation feeling.
B: Six to seven panelists evaluated as having permeation feeling.
C: Four to five panelists evaluated as having permeation feeling.
D: Two to three panelists evaluated as having permeation feeling.
E: One or fewer panelists were evaluated as having permeation feeling.

### [Evaluation of Usability: Slimy Feeling, Squeaky Feeling, and Moisturizing Feeling]

The cosmetic prepared above was applied to the skin by ten expert panelists and slimy feeling during application, squeaky feeling, and moisturizing feeling after application of the cosmetic were evaluated.

Here, the “skin blending” means that the cosmetic spreads over the skin, fits without discomfort, and provides a feeling that the application action is finished. “Smooth” means that the cosmetic does not give a dragging sensation on the skin when the cosmetic is applied to the skin. In addition, “firmness feeling” means feeling of elasticity on the skin after the cosmetic is applied.
A+: Ten panelists evaluated as having no slimy feeling/no squeaky feeling/moisturizing feeling.
A: Eight to nine panelists evaluated as having no slimy feeling/no squeaky feeling/moisturizing feeling.
B: Six to seven panelists evaluated as having no slimy feeling/no squeaky feeling/moisturizing feeling.
C: Four to five panelists evaluated as having no slimy feeling/no squeaky feeling/moisturizing feeling.
D: Two to three panelists evaluated as having no slimy feeling/no squeaky feeling/moisturizing feeling.
E: One or fewer panelists evaluated as having no slimy feeling/no squeaky feeling/moisturizing feeling.

The obtained results are also shown in Table 1 below.

TABLE 1

| | | Com. | | | | | | | | | | | |
|---|---|---|---|---|---|---|---|---|---|---|---|---|---|
| | | Ex | Example | | | | | | | | | | |
| | | 1 | 1 | 2 | 3 | 4 | 5 | 6 | 7 | 8 | 9 | 10 | 11 |
| | Water | balance | balance | balance | balance | balance | balance | balance | balance | balance | balance | balance | balance |
| A | Bakuchiol | — | 0.2 | 1.5 | 0.2 | 0.2 | 1.8 | 0.1 | 0.2 | 0.2 | 1.5 | 0.2 | 0.2 |
| B | Diglycerin | 1 | 1 | 1 | 0.3 | 5 | 0.3 | 1.5 | 1 | 1 | 1 | 1 | 1 |
| C | (Acrylate/alkyl | — | — | — | — | — | — | — | 0.1 | 0.3 | 0.1 | — | — |
| | acrylate (C10-30)) | | | | | | | | | | | | |
| | crosspolymer | | | | | | | | | | | | |
| D | Xanthan gum | — | — | — | — | — | — | — | — | — | — | 0.1 | — |
| E | Allantoin | — | — | — | — | — | — | — | — | — | — | — | 0.2 |
| | Beheneth-20 | 0.3 | 0.3 | 0.3 | 0.3 | 0.3 | 0.3 | 0.3 | 0.3 | 0.3 | 0.3 | 0.3 | 0.3 |
| | Triethylhexanoin | 1 | 1 | 1 | 1 | 1 | 1 | 1 | 1 | 1 | 1 | 1 | 1 |
| | Isododecane | 1 | 1 | 1 | 1 | 1 | 1 | 1 | 1 | 1 | 1 | 1 | 1 |
| | Squalane | 1 | 1 | 1 | 1 | 1 | 1 | 1 | 1 | 1 | 1 | 1 | 1 |
| | BG | 5 | 5 | 5 | 5 | 5 | 5 | 5 | 5 | 5 | 5 | 5 | 5 |
| | Potassium hydroxide | q.s. | q.s. | q.s. | q.s. | q.s. | q.s. | q.s. | q.s. | q.s. | q.s. | q.s. | q.s. |
| | Phenoxyethanol | q.s. | q.s. | q.s. | q.s. | q.s. | q.s. | q.s. | q.s. | q.s. | q.s. | q.s. | q.s. |
| | EDTA-3Na | q.s. | q.s. | q.s. | q.s. | q.s. | q.s. | q.s. | q.s. | q.s. | q.s. | q.s. | q.s. |
| | Total | 100 | 100 | 100 | 100 | 100 | 100 | 100 | 100 | 100 | 100 | 100 | 100 |
| | B/A | — | 5 | 0.67 | 1.5 | 25 | 0.17 | 15 | 5 | 5 | 0.67 | 5 | 5 |
| | C/A | — | — | — | — | — | — | — | 0.5 | 1.5 | 0.07 | — | — |
| Evaluation | Permeation feeling | E | A | A | A | A | B | B | A+ | A | A+ | A | A |
| | Slimy feeling | E | A | A | A | B | A | A | A | A | A | A+ | A+ |
| | Squeaky feeling | E | A | A | A | B | A | B | A+ | A+ | A+ | A | A |
| | Moisturizing feeling | B | A | A | A | A | B | A | A | A | A | A+ | A+ |

1. A cosmetic comprising:
(A) a bakuchiol; and
(B) polyglycerin.

2. The cosmetic according to claim 1, further comprising: (C) an alkyl-modified carboxyvinyl polymer.

3. The cosmetic according to claim 1, further comprising: (D) a polysaccharide.

4. The cosmetic according to claim 1, further comprising: (E) a carboxamide derivative.

5. The cosmetic according to claim 1, wherein component (B) comprises diglycerin.

6. The cosmetic according to claim 2, wherein component (C) comprises an (acrylate/alkyl acrylate (C10-30)) crosspolymer.

7. The cosmetic according to claim 3, wherein component (D) comprises xanthan gum.

8. The cosmetic according to claim 4, wherein component (E) comprises allantoin.

9. The cosmetic according to claim 1, wherein a mass ratio B/A of a content amount of component (B) to a content amount of component (A) contained in said cosmetic is 0.01 to 100.

10. The cosmetic according to claim 2, wherein a ratio C/A of a content amount of component (C) to a content amount of component (A) contained in said cosmetic is 0.01 to 20.

11. The cosmetic according to claim 1, which is an oil-in-water cosmetic.
